(19) United States
(12) Patent Application Publication
(10) Pub. No.: US 2026/0263695 A1
(43) Pub. Date: Sep. 10, 2026

(54) DEVICE FOR ADMINISTERING A FLUID SUBSTANCE

(71) Applicant: Ypsomed AG, Burgdorf (CH)

(72) Inventors: Oliver Glauser, Bern (CH); Dominik Zumstein, Lyss (CH); Simon Martin Bosshard, Hindelbank (CH); Christian Schrul, Oberburg (CH); Markus Tschirren, Burgdorf (CH); Peter Loser, Herrenschwanden (CH)

(21) Appl. No.: 19/661,347

(22) Filed: Apr. 28, 2026

Related U.S. Application Data
(63) Continuation of application No. PCT/EP2024/079495, filed on Oct. 18, 2024.

(30) Foreign Application Priority Data
Oct. 31, 2023 (EP) 23207091.2

(51) Int. Cl. A61M 5/20; A61M 5/28; A61M 5/32

(52) U.S. Cl. CPC A61M 5/20; A61M 5/28; A61M 5/3213; A61M 5/3245; A61M 5/326; A61M 2005/2013; A61M 2005/3267; A61M 2202/0007; A61M 2202/04; A61M 2207/00

(57) ABSTRACT

A device and a method for assembling this device for administering a fluid substance are provided. The device includes a housing and a product container, which is accommodated in an axially fixed manner in the housing and has a needle, where the substance can be administered through the needle. The device includes an inwardly directed shoulder support, which is provided on the housing and on which the product container bears with a distally arranged shoulder, in order to prevent an axial relative movement between the product container and the housing in the distal direction.

### CROSS-REFERENCE TO RELATED APPLICATIONS

This application is a continuation of International Patent Application No. PCT/EP2024/079495, filed Oct. 18, 2024, entitled “DEVICE FOR ADMINISTERING A FLUID SUBSTANCE,” which in turn claims priority to European Patent Application No. 23207091.2, filed Oct. 31, 2023, entitled “DEVICE FOR ADMINISTERING A FLUID SUBSTANCE”, each of which is incorporated by reference herein, in the entirety and for all purposes.

### TECHNICAL FIELD

Implementations relate to a device having a longitudinal axis (L) and to the assembly of this device for administering a fluid substance, in particular a medicament.

### BACKGROUND

Devices for administering a fluid substance are known from the prior art. From EP 2742962 B1, for example, an autoinjector with a housing and a syringe is known, wherein the syringe is accommodated in an axially fixed manner in a syringe holder. The syringe holder has a shoulder support which serves to secure the syringe at one shoulder against movement along the longitudinal axis (L) in the distal direction relative to the syringe holder. In addition, the housing has a retaining portion which engages the shoulder support of the syringe holder with the shoulder of the syringe to secure the syringe in the autoinjector against movement along the longitudinal axis (L) in the distal direction.

A disadvantage of this autoinjector is its complex design.

### SUMMARY

It is an object of the present disclosure to provide an alternative device and a method of assembling this device for administering a fluid substance, which is simple and safe in design.

The device according to the present disclosure with a longitudinal axis (L) for administering a fluid substance, in particular a medicament, has a housing. The casing may be elongated.

The housing may be sleeve-shaped and/or cylindrical, in particular circular cylindrical. A product container is accommodated in an axially fixed manner within the housing. The product container includes the substance to be administered. The product container has a needle through which the substance can be administered. The product container can be designed as a syringe with a syringe body and a needle accommodated at a distal end of the syringe body. Alternatively, the product container can be designed as a cartridge with a cartridge body and an attachable and removable needle unit. The needle or needle unit may have a removable needle guard to protect the needle from contamination and to keep the needle and the substance to be administered sterile.

The device according to the present disclosure further includes an inwardly directed shoulder support provided on the housing, against which the product container rests with a distally arranged shoulder in order to prevent axial relative movement between the product container and the housing in the distal direction. The shoulder of the product container is designed as a tapered portion in the distal region of the product container, in particular in the distal region of the syringe body or the cartridge body. The inward-facing shoulder support of the housing may be elastic, in particular may be an elastic retaining arm or elastic snap arm. One or more holding arms or snap arms may be provided. The shoulder support may be configured in such a way that it can engage with the shoulder of the product container to prevent axial relative movement between the product container and the housing in the distal direction. The shoulder support of the housing is designed to be radially pre-tensioned outward or radially deflectable. The needle of the product container is covered with the removable needle guard sleeve, with the inward-facing shoulder support of the housing snapping or engaging between a proximal end of the needle guard sleeve and the distal shoulder of the product container when the product container is accommodated in an axially fixed manner in the housing. In the engagement position, the shoulder support is either relaxed or snapped into place, with the shoulder of the product container resting against the shoulder support of the housing. The shoulder support of the housing may be formed in one piece or integrally with the housing. This makes it possible to construct the device with few components and at low cost, because the use of the product container holder for holding and inserting the product container into the housing of the device can be eliminated during assembly of the device. Alternatively, the shoulder support and the housing can be designed in two parts or as two pieces, with the shoulder support being arranged axially and rotationally fixed to the housing. This simplifies the assembly of the device.

Furthermore, the device according to the present disclosure may include a needle guard element that is movable relative to the housing. The needle guard element can be sleeve-shaped and/or cylindrical, in particular circular-cylindrical. The needle guard element can serve to protect against needlestick injuries, in particular from a distal tip of the needle. Furthermore, the device has a needle guard spring, which is coupled to the needle guard element, where the needle guard element is axially movable from a starting position, in which the distal tip of the needle is surrounded by the needle guard element, and into a release position, in which the distal tip of the needle is released from the needle guard element. From the starting position to the release position, the needle guard element moves in the proximal direction relative to the housing.

Furthermore, the device according to the present disclosure may include a drive device, which is driven into a discharge position by a discharge spring in order to administer the substance from the product container. The discharge spring provides the energy to effect the substance administration, in order to administer the substance from the product container through the needle. The device can be designed as an autoinjector. Autoinjectors are often described as devices that automatically deliver a substance to be administered to a patient after activation. For instance, the drive device can be released or freed by the axial movement of the needle guard element from the starting position to the release position in order to administer the substance from the product container.

The needle guard element may include a cam or a projection, which, in the initial position of the needle guard element, engages with an opening in the housing or in a housing-mounted part or abuts with a distal end of an opening in the housing or in a housing-mounted part, where the cam or the projection is elastically arranged on the needle guard element. The cam or projection can be arranged to be deflected radially or tangentially. Alternatively, a cam or a projection can be provided on the housing or on a housing-fixed part which engages with an opening in the needle guard element or abuts with a distal end of an opening on the needle guard element, where the cam or projection is elastically arranged on the housing or on a housing-mounted part. This engagement or stop serves to form a release resistance between the needle guard element and the housing or a housing-mounted part of the device. When the needle guard element moves from the starting position to the release position, a release resistance must be overcome, which at least makes unintentional release more difficult.

Alternatively, the device can be triggered by pressing a release button. The device can be designed in such a way that manual or automatic insertion of the needle into the patient's skin and/or manual or automatic retraction of the needle into the housing of the device is provided.

The needle guard spring and the discharge spring can be designed as compression springs. The two springs may be helical springs. However, other springs may also be provided. The needle guard spring and the discharge spring may be configured to be tensioned and released. The force of the needle guard spring may cause the needle guard spring to move the needle guard element from the starting position to the discharge position and back in the distal direction to a protective position. The force of the discharge spring allows the discharge spring to discharge the entire amount of the substance to be administered or a partial amount of the substance to be administered. The needle guard spring and the discharge spring may be made of metal. Other materials, such as plastics, may also be provided.

The device according to the present disclosure may further include a compensating spring provided on the housing, for instance supported at a proximal end of the product container and by means of which the product container is elastically supported on the housing in and counter to the direction of discharge. The compensating spring can be formed in one piece or integrally with the housing. This makes it possible to construct the device with few components and at low cost, because the use of the device's compensating spring can be dispensed with during assembly. Alternatively, the compensating spring and the housing can be designed in two parts or as two pieces, where the compensating spring and the housing are fixed axially and rotationally to each other. This makes it possible to easily assemble the device. The compensating spring is designed to be tensioned and released. The compensating spring is designed in such a way that the compensating spring is supported at the proximal end of the product container and that the product container is elastically supported against the housing in and counter to the direction of discharge via the compensating spring. This allows for compensation of length differences in the product container that may arise due to manufacturing tolerances. Furthermore, the secure fit of the product container to the shoulder support of the housing can be ensured. The compensating spring may be made of plastic. Other materials, such as metal, may also be used.

The needle guard element can, in a distal position, allow radial outward pre-tensioning or radial deflection of the shoulder support of the housing, and, in a proximal position, prevent radial outward pre-tensioning or radial deflection of the shoulder support of the housing. This allows the product container to be inserted axially into the housing in a distal direction, and also prevents axial relative movement between the product container and the housing in the distal direction when the product container is held in an axially fixed manner in the housing. The needle guard element keeps the shoulder support of the housing engaged with the shoulder of the product container to secure the product container in the device against movement along the longitudinal axis (L) in the distal direction. The needle guard element is shaped like a sleeve. The needle guard element may be arranged radially between the product container and the housing. The needle guard element may have a wall thickness along the longitudinal axis (L). The wall thickness of the needle guard element can vary along the longitudinal axis (L). The wall thickness of the needle guard element is determined such that radial deflection of the shoulder support is possible during one assembly step of the device, and radial deflection of the shoulder support is prevented during another assembly step of the device. The mounting position of the device depends on the axial relative position between the needle guard element and the housing. For instance, a distal region of the needle guard element may have a greater wall thickness than a proximal region of the needle guard element. A proximal region of the needle guard element may have a thinner wall thickness than a distal region of the needle guard element. The needle guard element can include a distal and a proximal region, which have different wall thicknesses.

The present disclosure further relates to a method for assembling this device. As a first step, a housing with an inward-facing shoulder support is provided to accommodate a product container. A needle guard element is pre-installed in the housing. The needle guard element is positioned in a distal position relative to the housing. Furthermore, a product container with a distally arranged shoulder and a needle covered with a removable needle guard is provided. In a further step, the product container is moved or inserted into the housing along a longitudinal axis (L) in a distal direction. During this axial relative movement between the product container and the housing, in particular during this displacement or during this insertion of the product container into the housing, the shoulder support of the housing is positioned in the proximal region of the needle guard element. The needle guard element is positioned in the distal position relative to the housing. In the distal position of the needle guard element, the proximal region of the needle guard element is radially offset relative to the shoulder support of the housing. In the distal position of the needle guard element, the shoulder support of the housing can be pre-tensioned radially outward or deflected radially. During the insertion of the product container into the housing, the inward-facing shoulder support of the housing slides over an outer surface of the needle guard sleeve of the product container and is thereby radially pre-tensioned outward or radially deflected. If the radially outwardly pre-tensioned or radially deflected shoulder support of the housing comes into contact with or is positioned between a proximal end of the needle guard sleeve and the distally located shoulder of the product container, the radially outwardly pre-tensioned or radially deflected shoulder support may relax or snap into place between the proximal end of the needle guard sleeve and the distally located shoulder. The needle guard element can then be moved relative to the housing in the proximal direction up to a proximal position. In the proximal position of the needle guard element, a renewed radial outward pre-tensioning or a renewed radial deflection of the shoulder support of the housing is prevented by the needle guard element. In the proximal position of the needle guard element, the distal region of the needle guard element is radially offset relative to the shoulder support of the housing. The proximal position of the needle guard element may form the starting position of the needle guard element.

The advantage of this method is that few assembly steps are required to create the device according to the present disclosure. During assembly, it is not necessary to provide a product container holder for holding and inserting the product container into the housing of the device. This allows for the provision of a cost-effective device in accordance with the present disclosure.

Furthermore, the housing of the device according to the present disclosure can have a housing stop and the needle guard element can have a needle guard stop, where when in contact with the stop, the needle guard element is in the proximal position or a stop position of the device. The housing stop and/or the needle guard stop can be designed to be elastic. In the proximal or stop position of the device, the needle guard element can no longer be pushed further relative to the housing and into the housing. This ensures that radial outward pre-tensioning or radial deflection of the shoulder support of the housing is prevented. The product container is supported by its distally positioned shoulder on the inwardly facing shoulder support of the housing to prevent axial relative movement between the product container and the housing in the distal direction.

In a further step, a compensating spring, which is supported at a proximal end of the product container and by means of which the product container is elastically supported against the housing in and counter to the direction of discharge, can be mounted in or on the housing. In an alternative embodiment, the housing already has this compensating spring. The compensating spring serves to ensure that the product container is firmly seated on the shoulder support of the housing.

In order for the patient to use the device to administer the substance, the patient must first remove a cap that is detachably attached to the device. Removing the cap from the device removes the removable needle guard attached to the needle. The patient then presses the device against the skin, where the needle guard element is moved from the starting position to the release position relative to the housing in the proximal direction.

If a certain threshold of the force acting on the stop in the proximal direction is exceeded, the housing stop or the needle guard stop can be deflected radially or tangentially, so that the needle guard element can be moved into the release position in which a distal tip of the needle is released.

Alternatively or additionally, if a certain threshold of the force acting in the proximal direction on the stop or engagement is exceeded, the trigger resistance already described can be overcome.

The needle is inserted into the patient's skin during or as the needle guard element moves into the release position. The needle guard element moves from the starting position to the release position, releasing the drive mechanism to administer the substance from the product container. The drive unit is driven into the discharge position by a discharge spring in order to administer the substance from the product container. At the end of the administration, the patient can remove the device from the skin. The force of the needle guard spring moves the needle guard element from the release position in the distal direction into the guard position. The starting position and the protective position can be provided along the longitudinal axis (L) of the device at the same axial position or at different axial positions. In the protective position, the needle guard element can be locked, thus preventing the needle guard element from moving back into the proximal position. The patient cannot trigger the device again.

### BRIEF DESCRIPTION OF THE DRAWINGS

Implementations of the present disclosure will be described below with reference to a number of drawings. Both individually and in combination, the features disclosed herein advantageously develop the implementations further. In the drawings:

FIG. 1 shows a perspective longitudinal section view of an embodiment of the device according to the present disclosure with a longitudinal axis (L), in a first assembly step.

FIG. 2 shows a perspective longitudinal section view of the device according to FIG. 1 in a second assembly step.

FIG. 3 shows a perspective longitudinal section view of the device according to FIG. 1 in a third assembly step.

### DETAILED DESCRIPTION

### Definitions

The term “medicament” as used herein comprises any flowable medical formulation suitable for controlled administration through a means such as a cannula or needle, for example comprising a liquid, a solution, a gel, or a fine suspension containing one or more active medical ingredients. A medicament can be a single active substance composition or a pre-mixed or co-formulated composition, having multiple active substances, from a single container. Medicament comprises drugs such as peptides (e.g., insulins, insulin-containing medications, GLP-1-containing and derived or analogous preparations), proteins and hormones, biologically obtained or active ingredients, active ingredients based on hormones or genes, nutrient formulations, enzymes and further substances both in solid (suspended) or liquid form but also polysaccharides, vaccines, DNS or RNS or oligonucleotides, antibodies or parts of antibodies, and suitable base, auxiliary and excipient substances.

The proximal direction of a device for administering a fluid substance means the direction toward the housing-side end, and the distal direction of this device means the direction toward the needle-side end.

### Detailed Description of the Figures

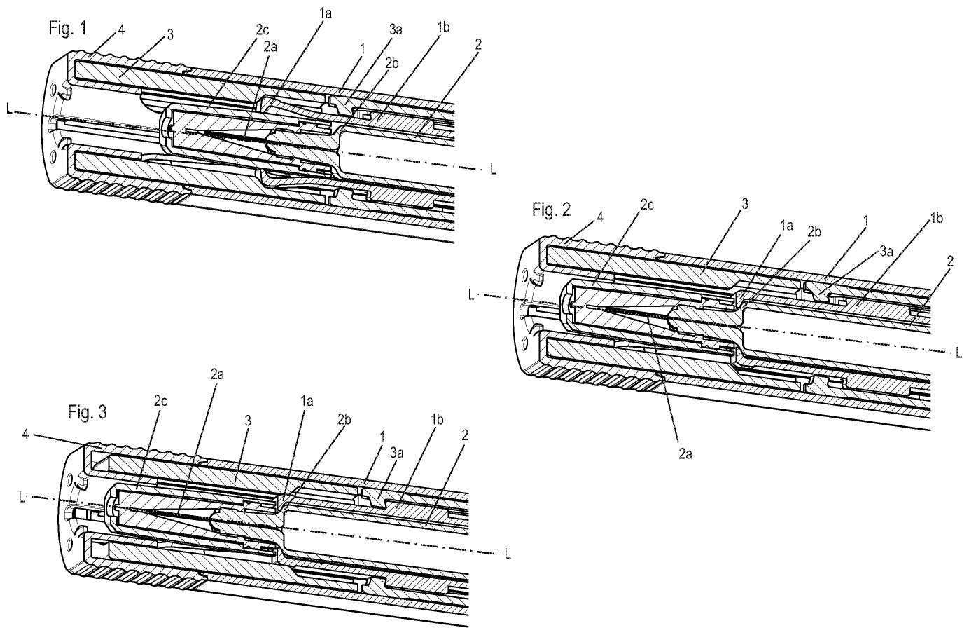

FIG. 1 shows a perspective longitudinal sectional view of an embodiment of the device according to the present disclosure with a longitudinal axis (L) in a first assembly step. First, a housing (1) with an inwardly directed shoulder support (1a) is provided to accommodate a product container (2). The inward-facing shoulder support (1a) is elastically designed. The housing (1) and the inwardly directed shoulder support (1a) are formed in one part or one piece. Furthermore, the product container (2) is provided with a distally arranged shoulder (2b) and a needle (2a) which is covered with a removable needle guard sleeve (2c). A needle guard element (3) is arranged radially between the product container (2) and the housing (1). The needle guard element (3) is arranged in a distal position. The needle guard element (3) has a different wall thickness along the longitudinal axis (L). The wall thickness of the needle guard element (3) is determined such that radial deflection of the shoulder support (1a) is possible in one assembly step of the device and radial deflection of the shoulder support (1a) is prevented in another assembly step of the device. A distal region of the needle guard element (3) has a greater wall thickness than a proximal region of the needle guard element (3). The proximal region of the needle guard element (3) has a thinner wall thickness than the distal region of the needle guard element (3).

As can be seen in FIG. 2, in a second assembly step, the product container (2) is moved or inserted into the housing (1) along a longitudinal axis (L) in a distal direction. During this axial relative movement between the product container (2) and the housing (1), the inwardly directed shoulder support (1a) of the housing (1) slides over an outer surface of the needle guard sleeve (2c). The shoulder support (1a) of the housing (1) is thereby pre-tensioned radially outward or deflected radially. The proximal region of the needle guard element (3) is arranged radially offset relative to the shoulder support (1a) of the housing (1), whereby radial outward pre-tensioning or radial deflection of the shoulder support (1a) of the housing (1) is possible. When the radially outwardly pre-tensioned or radially deflected shoulder support (1a) of the housing (1) arrives or is positioned between a proximal end of the needle guard sleeve (2c) and the distally arranged shoulder (2b) of the product container (2), the radially outwardly pre-tensioned or radially deflected shoulder support (1a) can relax or snap into place between the proximal end of the needle guard sleeve (2c) and the distally arranged shoulder (2b). When the shoulder support (1a) of the housing (1) is snapped between the proximal end of the needle guard sleeve (2c) and the distally arranged shoulder (2b) of the product container (2), the shoulder support (1a) of the housing (1) is relaxed. The proximal region of the needle guard element (3) is arranged between the product container (2) and the housing (1).

As shown in FIG. 3, in a third assembly step, the needle guard element (3) is moved relative to the housing (1) and into the housing (1) in the proximal direction up to a proximal position of the needle guard element (3). In the proximal position of the needle guard element (3), a renewed radial outward pre-tensioning or a renewed radial deflection of the shoulder support (1a) of the housing (1) is prevented. The distal region of the needle guard element (3) is arranged radially offset relative to the shoulder support (1a) of the housing (1). In this position, radial deflection or radial swiveling of the shoulder support (1a) of the housing (1) is prevented by the axial arrangement of the needle guard element (3). In the proximal position of the needle guard element (3), the needle guard element (3) holds the shoulder support (1a) of the housing (1) in engagement with the shoulder (2b) of the product container (2). A needle guard stop (3a) provided on the needle guard element (3) comes into contact with a housing stop (1b) attached to the housing (1). The stop forms and/or defines the stop position of the device and/or the proximal position of the needle guard element (3). The housing stop (1b) and/or the needle guard stop (3a) can be designed to be elastic. In the stop position of the device, the needle guard stop is in contact with the housing stop, so that the needle guard element can no longer be pushed further relative to the housing (1) and into the housing (1). The product container (2) is supported by its distally arranged shoulder (2b) on the inwardly directed shoulder support (1a) of the housing (1) in order to prevent axial relative movement between the product container (2) and the housing (1) in the distal direction.

In a further assembly step, a compensating spring—which is supported at a proximal end of the product container (2) and by means of which the product container (2) is elastically supported on the housing (1) in and counter to the direction of discharge—can be fixedly mounted on or in the housing (1). The compensating spring serves to ensure that the product container (2) is securely seated on the shoulder support (2b) of the housing (1).

In order for the patient to use the device to administer the substance, the patient must first remove a cap (4), which is detachably arranged on the device. Removing the cap (4) from the device causes the needle guard (2c), which is removable from the needle (2a) to be removed. The patient then presses the device against the skin, where the needle guard element (3) is moved from the distal position to the proximal position relative to the housing (1). If a certain threshold value of the force acting in the proximal direction is exceeded, in particular the force acting on the housing stop (1b) of the housing (1) via the needle guard element (3), the housing stop (1b) is deflected radially or tangentially, so that the needle guard element (3) is moved into a proximal position in which a distal tip of the needle (2a) is exposed. The needle guard element (3) moves from the distal position to the proximal position, releasing a drive device to administer the substance from the product container (2). The drive unit is driven into a discharge position by a discharge spring in order to administer the substance from the product container (2). At the end of the administration, the patient can remove the device from the skin. The force of the needle guard spring moves the needle guard element (3) in the distal direction. The needle guard element (3) is locked in place, preventing further movement of the needle guard element (3) in the proximal direction. The patient cannot initiate the procedure again.

### REFERENCE SIGNS

1 housing
1a shoulder support
1b housing stop
2 product container
2a needle
2b shoulder
2c needle guard sleeve
3 needle guard element
3a needle guard stop
4 cap
L longitudinal axis

What is claimed is:

1. A device for administering a fluid substance with a longitudinal axis (L) comprising:
a housing;
a product container accommodated in the housing in an axially fixed manner, the product container comprising a needle through which the substance can be administered;
an inwardly directed shoulder support provided on the housing against which the product container is supported with a distally arranged shoulder thereof to prevent axial relative movement between the product container and the housing in a distal direction;
a needle guard element for protecting the needle, the needle guard element provided in and movable relative to the housing, wherein the needle guard element is axially movable against a spring force from an initial position in which a distal tip of the needle is surrounded by the needle guard element into a release position in which the distal tip of the needle is released from the needle guard element; and
a drive device movably arranged in the housing, wherein the drive device is driven into a discharge position to administer the substance from the product container,
wherein the product container is configured to be elastically supported at a proximal end on the housing in and counter to a direction of discharge to thereby accommodate the product container in the housing in the axially fixed manner.

2. The device according to claim 1, wherein the needle guard element, in a distal position, prevents radial outward pre-tensioning or radial deflection of the shoulder support, a proximal region of the needle guard element being arranged radially offset relative to the shoulder support of the housing.

3. The device according to claim 1, wherein the needle guard element, in a proximal position, prevents radial outward pre-tensioning or radial deflection of the shoulder support, a distal region of the needle guard element being arranged radially offset relative to the shoulder support of the housing.

4. The device according to claim 1, wherein the drive device is released by the axial movement of the needle guard element from the starting position to the release position to administer the substance from the product container.

5. The device according to claim 1, wherein the needle of the product container is covered with a removable needle guard sleeve and the shoulder support of the housing is snapped or engages between a proximal end of the needle guard sleeve and the distal shoulder of the product container.

6. The device according to claim 1, wherein the shoulder support is formed integrally with the housing.

7. A method for assembling the device according to claim 1, comprising the following steps:
providing the housing with the shoulder support for receiving the product container;
providing the product container with the distally arranged shoulder and the needle, which is covered with a removable needle guard sleeve;
moving or inserting the product container into the housing along the longitudinal axis in the distal direction, wherein the shoulder support slides over an outer surface of the needle guard sleeve and is thereby radially outwardly pre-tensioned or radially deflected;
relaxing or snapping in the radially outwardly pre-tensioned or radially deflected shoulder support between a proximal end of the needle guard sleeve and the distally arranged shoulder of the product container; and
moving the needle guard element relative to and into the housing in a proximal direction to a proximal position of the needle guard element or to a stop position of the device, which prevents a second radial outward pre-tensioning or a second radial deflection of the shoulder support.

8. The method according to claim 7, wherein in the proximal position of the needle guard element or in the stop position of the device, the needle guard element is in contact with a needle guard stop against a housing stop of the housing.

9. The method according to claim 8, wherein when a certain threshold value of the force acting on the stop via the needle guard element in the proximal direction is exceeded, the housing stop or the needle guard stop is deflected radially, such that the needle guard element can be moved into a release position in which a distal tip of the needle is released.

10. The method according to claim 9, wherein when the needle guard element is moved from a starting position to the release position, the drive device is released to eject the substance from the product container.

11. The method according to claim 10, wherein the drive device is driven into the discharge position to eject the substance from the product container.

12. A device for ejecting a substance comprising:
a housing comprising an integrally-formed, flexible, inwardly directed shoulder support for receiving a product container;
the product container comprising a distally arranged shoulder and a needle which is covered with a removable needle guard sleeve,
wherein the product container is adapted to be moved or inserted into the housing along a longitudinal axis in a distal direction such that the inwardly directed shoulder support slides over an outer surface of the needle guard sleeve and is thereby outwardly radially deflected and relaxed into a gap between a proximal end of the needle guard sleeve and the distally arranged shoulder of the product container; and
a needle guard element adapted to be moved relative to and into the housing in the proximal direction to a first proximal position to prevent the outward radial deflection of the inwardly directed shoulder support.

13. The device according to claim 12, wherein in the first proximal position of the needle guard element, a needle guard stop of the needle guard element abuts against a housing stop of the housing.

14. The device according to claim 13, wherein when a certain threshold value of a force acting on the housing stop via the needle guard element in the proximal direction is exceeded, the housing stop or the needle guard stop is deflected radially such that the needle guard element is movable from the first proximal position into a release position in which a distal tip of the needle is released.

15. The device according to claim 14, wherein when the needle guard element is moved to the release position, a drive device is released to eject the substance from the product container.

16. The device according to claim 15, wherein the drive device is driven into a discharge position to eject the substance from the product container.
